(19) United States
(12) Patent Application Publication
(10) Pub. No.: US 2021/0196589 A1
(43) Pub. Date: Jul. 1, 2021

(54) COMPOSITIONS, DISPENSING SYSTEMS AND METHODS FOR PROVIDING SENSORIAL AND AESTHETIC BENEFITS TO ACNE PRONE SKIN

(71) Applicant: L'OREAL, Paris (FR)

(72) Inventors: Stephanie Anne Venture VENTURE MORRIS, Montclair, NJ (US); Nicholas David STEBBINS, Clark, NJ (US)

(73) Assignee: L'OREAL, Paris (FR)

(21) Appl. No.: 16/727,906

(22) Filed: Dec. 26, 2019

(51) Int. Cl. A61K 8/34; A61K 8/25; A61K 8/02; A61K 8/04; A61Q 19/00; A45D 34/04; A61K 8/81

(52) U.S. Cl. CPC A61K 8/342; A61K 8/25; A61K 8/0279; A61K 2800/87; A61Q 19/00; A45D 34/04; A61K 8/8152; A61K 8/042

(57) ABSTRACT

A cosmetic composition includes at least one beta hydroxy acid selected from -n-octanoylsalicylic acid, salicylate, sodium salicylate, willow extract, beta hydroxybutanoic acid, tropic acid, and trethocanic acid present in a hydroalcoholic gel dispersion that includes at least one C2-C5 alcohol, (ii) a blend of at least two powders selected from inorganic powders and organic powders, and (iii) at least one polymer. The cosmetic composition may be dispensed using a porous applicator and is applied as an on-the-go skin refresher that is especially good for acne-prone skin. The cosmetic composition can be used throughout the day to control shine and minimize skin imperfections and discourage acne and is suitable for use alone or over or under make up.

### FIELD OF THE INVENTION

The present disclosure is directed to skin care compositions cosmetic dispensing systems, and methods that provide sensorial and aesthetic benefits to acne prone skin. The benefits include shine and acne control, in a light and refreshing cooling gel that can be applied alone, under or over makeup.

### BACKGROUND OF THE INVENTION

Prior art compositions that have a low pH and high salt content, such as hydroxy acid-containing compositions, typically employ one or more of surfactants and high amounts of polymeric thickeners to maintain stability and consistency while maintaining the solubility of the acid. Inclusion of emulsifying surfactants and high amounts of polymeric thickeners can be associated with tackiness, unwanted whitening upon application, and with skin drying and irritation and overall loss of skin hydration. In addition to the forgoing, compositions that employ surfactants and high amounts of polymeric thickeners do not provide a fresh skin feel, do not diminish oiliness or minimize skin imperfections, and cannot be conveniently applied over or under makeup.

Accordingly, there is a need for a composition that overcomes the shortcomings of the prior art and provides benefits that include the ability to incorporate amounts of soluble hydroxy acids, for example salicylic acid, effective for treating acne, in a stable gel dispersion of oil absorbing powders, without emulsifying surfactants and without high content of polymers.

### BRIEF SUMMARY OF THE INVENTION

In accordance with the various embodiments, provided are compositions, cosmetic dispensing systems, and methods that provide sensorial and aesthetic benefits to acne prone skin. The benefits include shine and acne control, in a light and refreshing cooling gel that can be applied alone, under or over makeup.

In various embodiments, the disclosure provides a cosmetic composition that includes at least one beta hydroxy acid selected from -n-octanoylsalicylic acid, salicylate, sodium salicylate, willow extract, beta hydroxybutanoic acid, tropic acid, and trethocanic acid. The at least one beta hydroxy acid is present in a hydroalcoholic gel dispersion. The hydroalcoholic gel dispersion includes at least one C2-C5 alcohol, (ii) a blend of at least two powders selected from inorganic powders and organic powders, and (iii) at least one polymer.

In some embodiments, the at least one beta hydroxy acid is salicylic acid. In some embodiments, the at least one beta hydroxy acid comprises salicylic acid present in the composition in the range from about 0.2% to about 2%, by weight, based on the total weight of the composition.

In some embodiments, the at least one C2-C5 alcohol is selected from ethanol, propanol, butanol, pentanol, isopropanol, isobutanol and isopentanol. In some embodiments, the at least one C2-C5 alcohol comprises denatured alcohol present in the composition in the range from about 5% to about 20%, by weight, based on the total weight of the composition

In some embodiments, the pH of the cosmetic composition is in the range from about 3.9 to about 4.5.

In some embodiments, the blend of at least two powders comprises perlite, and one or more of silica silylate, and microcrystalline cellulose. In some embodiments, the blend of powders comprises perlite, silica silylate, and microcrystalline cellulose. In some embodiments, each one of the powders in the blend is present in the cosmetic composition in an amounts ranging from about 0.005% to about 10%, by weight based on the weight of the composition.

In some embodiments, the at least one polymer is not electrolyte sensitive or salt intolerant. In some embodiments, the at least one polymer is selected from Ammonium Acryloyldimethyltaurate/Steareth-25 Methacrylate Crosspolymer, and one of polyacrylate crosspolymer 6 and an acrylates/C10-30 Alkyl acrylate crosspolymer present from greater than about 1% by weight based on the weight of the composition. In some embodiments, the at least one polymer comprises Ammonium Acryloyldimethyltaurate/Steareth-25 Methacrylate Crosspolymer. In some embodiments, the at least one polymer excludes at least one or all of polyacrylate crosspolymer 6, and an acrylates/C10-30 Alkyl acrylate crosspolymer.

In some embodiments, the cosmetic composition has a gel crossover point ranging from about 35% to about 85% strain. In some embodiments, the cosmetic composition has a gel crossover point which allows it to suspend the at least one powder and pass through a porous applicator with shear.

In some embodiments, the cosmetic composition excludes one or more powders comprising Nylon-12, polymethyl methacrylate, polyethylene beads, and powders which are considered microplastics.

In some particular embodiments, the cosmetic composition comprises a hydroalcoholic gel dispersion, comprising (a) at least one beta hydroxy acid selected from -n-octanoylsalicylic acid, salicylate, sodium salicylate, willow extract, beta hydroxybutanoic acid, tropic acid, and trethocanic acid, the beta hydroxy acid present in the cosmetic composition in an amount that is up to about 2.0%; (b) a hydroalcoholic gel, comprising: (i) at least one C2-C5 alcohol, and (ii) at least one polymer that includes acryloyldimethyltaurate/steareth-25 methacrylate crosspolymer; and (c) at least one powder that includes silica silylate, and optionally one or more additional powders selected from inorganic powders, organic powders, and silica; wherein the at least one beta hydroxy acid is dissolved in the hydroalcoholic gel and the at least one powder is dispersed in the hydroalcoholic gel.

In some particular embodiments, the cosmetic composition includes: (a) salicylic acid present in an amount in the range from about 0.2% to about 2%, and an (b) aqueous hydroalcoholic gel dispersion that includes: (i) denatured alcohol, (ii) perlite, microcrystalline cellulose, and silica silylate, and (iii) acryloyldimethyltaurate/steareth-25 methacrylate crosspolymer.

In some particular embodiments, the cosmetic composition includes (a) salicylic acid present in an amount in the range from about 0.2% to about 2%, and an (b) aqueous hydroalcoholic gel dispersion, comprising: (i) denatured alcohol in an amount that is about 20% by weight, (ii) perlite present in the cosmetic composition in an amount that ranges from about 0.5% to about 1% by weight, microcrystalline cellulose present in the cosmetic composition in an amount that ranges from about 1.4% to about 2% by weight, and silica silylate present in the cosmetic composition in an amount that ranges from about 0.2% to about 0.3% by weight, and (iii) acryloyldimethyltaurate/steareth-25 methacrylate crosspolymer present in the cosmetic composition in an amount that ranges from about 0.9% to about 2.4% by weight.

In some embodiments, the invention also relates to a cosmetic dispensing system for addressing shine and acne that includes a cosmetic composition and a dispenser.

In various embodiments, the disclosure provides a cosmetic dispensing system that includes, a cosmetic composition that has a gel crossover point suitable for dispensing through a porous applicator, the wherein the cosmetic composition comprising: at least one beta hydroxy acid selected from -n-octanoylsalicylic acid, salicylate, sodium salicylate, willow extract, beta hydroxybutanoic acid, tropic acid, and trethocanic acid, and a hydroalcoholic gel dispersion, the hydroalcoholic gel dispersion comprising: at least one C2-C5 alcohol; a blend of at least two powders selected from inorganic powders and organic powders, and at least one polymer. The cosmetic dispensing system further includes a dispenser that includes a container that includes a retainer and an absorbent component disposed within the retainer, and a porous applicator that is in contact on a first side with the absorbent component in the retainer, and is exposed on a second side for contacting keratinous tissue to be treated, wherein the cosmetic composition is absorbed within the absorbent component and retained in the retainer, which retains the cosmetic composition in a dispersed gel state until dispensed; and wherein the dispenser optionally comprises a cap for covering the porous applicator when the dispensing system is not in use.

In some embodiments of the dispensing system, the cosmetic composition comprises a hydroalcoholic gel dispersion, comprising (a) at least one beta hydroxy acid selected from -n-octanoylsalicylic acid, salicylate, sodium salicylate, willow extract, beta hydroxybutanoic acid, tropic acid, and trethocanic acid, the beta hydroxy acid present in the cosmetic composition in an amount that is up to about 2.0%; (b) a hydroalcoholic gel, comprising: (i) at least one C2-C5 alcohol, and (ii) at least one polymer that includes acryloyldimethyltaurate/steareth-25 methacrylate crosspolymer; and (c) at least one powder that includes silica silylate, and optionally one or more additional powders selected from inorganic powders, organic powders, and silica; wherein the at least one beta hydroxy acid is dissolved in the hydroalcoholic gel and the at least one powder is dispersed in the hydroalcoholic gel.

In some embodiments, the porous applicator of the cosmetic dispensing system includes one or more of: on at least the exposed surface intended for contacting skin, a plurality of pores having an average size in a range from about 30 to about 200 microns; on at least the first surface intended for contacting the sponge containing the cosmetic composition in the retainer, a plurality of pores having an average size in a range from about 90 to 200 microns; from the first surface to the second surface throughout the porous applicator, a plurality of pores having an average size in a range from 1 to about 1000 microns; the porosity through the porous material from the first surface to the second surface is uniform and continuous; the porosity through the porous material from the first surface to the second surface is variable from larger pores at the first surface to smaller pores at the second surface; and the porosity through the porous material from the first surface to the second surface is variable from smaller pores at the first surface to larger pores at the second surface.

In some embodiments, the invention also relates to enhancing the appearance of a keratinous material comprising using the applicator of the cosmetic system to apply the cosmetic composition of the cosmetic system to keratinous material.

In various embodiments, the disclosure provides a method for enhancing the appearance of acne prone skin, comprising: providing a dispensing system comprising: a cosmetic composition that has a gel crossover point suitable for dispensing through a porous applicator, and a dispenser that includes a container that includes a retainer and an absorbent component disposed within the retainer, and a porous applicator that is in contact on a first side with the absorbent component in the retainer, and is exposed on a second side for contacting keratinous tissue to be treated, wherein the cosmetic composition comprises: at least one beta hydroxy acid selected from -n-octanoylsalicylic acid, salicylate, sodium salicylate, willow extract, beta hydroxybutanoic acid, tropic acid, and trethocanic acid, and a hydroalcoholic gel dispersion, the hydroalcoholic gel dispersion comprising: at least one C2-C5 alcohol; a blend of at least two powders selected from inorganic powders and organic powders, and at least one polymer; and, wherein the dispenser containing the cosmetic composition is used to apply the composition to skin that is prone to acne by utilizing the dispenser to apply the cosmetic composition by contacting the porous applicator to skin using a dabbing motion to dispense a light coating of the cosmetic composition onto select areas of the skin in dispensed doses that are in an amount that is from about 0.01 mg to about 100 mg per application stroke.

In some embodiments of the method, the cosmetic composition comprises a hydroalcoholic gel dispersion, comprising (a) at least one beta hydroxy acid selected from -n-octanoylsalicylic acid, salicylate, sodium salicylate, willow extract, beta hydroxybutanoic acid, tropic acid, and trethocanic acid, the beta hydroxy acid present in the cosmetic composition in an amount that is up to about 2.0%; (b) a hydroalcoholic gel, comprising: (i) at least one C2-C5 alcohol, and (ii) at least one polymer that includes acryloyldimethyltaurate/steareth-25 methacrylate crosspolymer; and (c) at least one powder that includes silica silylate, and optionally one or more additional powders selected from inorganic powders, organic powders, and silica; wherein the at least one beta hydroxy acid is dissolved in the hydroalcoholic gel and the at least one powder is dispersed in the hydroalcoholic gel.

It is to be understood that both the foregoing general description and the following detailed description are exemplary and explanatory only and are not restrictive of the invention.

### BRIEF DESCRIPTION OF THE DRAWINGS

In accordance with the various embodiments, provided is

Features and advantages of the general inventive concepts will become apparent from the following description made with reference to the accompanying drawings, including drawings represented herein in the attached set of figures, of which the following is a brief description:

FIG. 1 shows a prior art example of a porous dispenser;

FIG. 2 is a graphical representation of data obtained demonstrating salicylic acid solubility at various concentrations as polymer and ethanol content are varied;

FIG. 3 is a graphical representation of determining gel crossover point for an embodiment of the inventive cosmetic composition;

FIG. 4 is a graphical representation of determining gel crossover point for an embodiment of the inventive cosmetic composition;

FIG. 5 is a graphical representation of determining gel crossover point for an embodiment of a comparative experimental cosmetic composition;

FIG. 6 shows sweat resistance properties of inventive, comparative and control compositions;

FIG. 7 shows photographic images demonstrating powder penetration through porous applicator detected using cross- and plane-polarized microscopy obtained of various inventive and comparative compositions; and

FIG. 8 shows data from consumer test studies.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION OF THE INVENTION

“Free of surfactant” means compositions that are free of, or substantially free of, or devoid of a surfactant. Some specific but non-limiting examples of emulsifiers that are lacking from the cosmetic composition includes surfactants with a C10-C20 fatty alcohol or acid hydrophobe condensed with from about 2 to about 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan; mono- and di-C8-C20 fatty acids; polyoxyethylene sorbitan; alkyl polyglycosides and saccharide fatty amides (e.g. methyl gluconamides); alkyl ether sulfate and sulfonates; alkyl sulfates and sulfonates; alkylbenzene sulfonates; alkyl and dialkyl sulfosuccinates; C8-C20 acyl isethionates; C8-C20 alkyl ether phosphates; alkylethercarboxylate. Some specific emulsifiers that are lacking from the cosmetic composition include PEG-100 Stearate; PEG-20 Stearate and other esters of Poly(Ethylene Glycol); Sucrose Stearate and other emulsifiers based on sugar esters; Glyceryl Stearate and other glycerol esters; Disodium Ethylene Dicocamide PEG-15 Disulfate; Sodium Steroyl Glutamate and other fatty amides; Steareth-100 and other fatty ethers.

“Gel Crossover Point” (Sol/Gel Point), means the point at which the G″ (loss modulus) intersects the G′ (storage modulus), which is reported in % strain. G* (complex modulus) provides insight on the flexibility and stiffness of a material. The cross-over point indicates the amount of shear that is required to collapse the microstructure (G″>G′) and allow the material to flow. It is the point at which a composition transitions from a more solid state to a more liquid state, such as from a gel state to a liquid state. An example of a method for determining gel crossover point is as follows: a Discovery HR-2 Rheometer by TA Instruments may be used, having 40 mm parallel plate geometry on a stainless-steel flat Peltier plate. The test can be run @ 20° C., with test parameter of angular frequency of 1.0 rad/s and logarithmic sweep: Strain % 0.01 to 1000.0%. 5 points per decade. Results are reported in % strain.

“Keratinous materials” includes materials containing keratin such as hair, skin, scalp, eyebrows, lips and nails.

“Soluble” and “Solubility” as used herein in reference to beta hydroxy acid means that the beta hydroxy acid is and remains substantially or fully dissolved in the hydroalcoholic gel.

“Stable” and “Stability” as used herein means that the cosmetic composition does not show any signs of significant changes in one or more of appearance, color, odor, pH, or viscosity at room temperature over at least a one-month period.

The cosmetic composition, dispensing systems and methods can comprise, consist of, or consist essentially of the essential elements and limitations of the invention described herein, as well as any additional or optional ingredients, components, or limitations described herein or otherwise useful.

The cosmetic composition is an on-the-go skin refresher that is especially good for acne-prone skin. The cosmetic composition can be used throughout the day to control shine and minimize skin imperfections and discourage acne and is suitable for use alone or over or under make up.

The cosmetic composition overcomes shortcomings in the art pertinent to salicylic acid induced instability in cosmetic compositions. Unexpectedly, according to the various embodiments herein and as exemplified in this disclosure, a combination of powder and polymer in a hydroalcoholic gel dispersion provides a composition that maintains salicylic acid solubility with up to about 2% salicylic acid while stably suspending oil absorbing powders. And in various embodiments the cosmetic composition exhibits a gel crossover point which allows it to pass through a porous applicator. With reference to the prior art, for example United States Patent Application Publication Number US20190231663A1 provides a cooling touch up gel formulation for over make up application that can be applied using a porous applicator and that includes a hydroalcoholic gel dispersion of powders. As shown in the comparative examples herein, exemplified compositions shown therein cannot be suitably modified by adding salicylic acid without compromising the sensorial and sol-gel properties of the composition. More particularly, the exemplified compositions therein demonstrate instability which is visible as separation into layers upon the addition of salicylic acid as disclosed in the examples herein below.

In some embodiments, the cosmetic composition is formulated for use in a dispenser that includes a container that includes a retainer and an absorbent component disposed within the retainer, and a porous applicator that is in contact on a first side with the absorbent component in the retainer, and is exposed on a second side for contacting keratinous tissue to be treated. Together the cosmetic composition and the dispenser comprise a cosmetic dispensing system. The dispenser may further comprise a cap or lid for covering the porous applicator when the dispensing system is not in use. The cosmetic composition is absorbed and retained in a retainer, which includes in some embodiments a sponge, and retains the cosmetic composition in a dispersed gel state until dispensed for application to skin. The dispenser is actuated to dispense the cosmetic composition onto skin by transfer first from the sponge to the porous applicator and then passage through the porous applicator toward the outer surface by capillary action. In some embodiments, the actuation may be achieved by compression of the exposed side of the porous applicator. According to the process of dispensing, the cosmetic composition must convert from a gel to a liquid in order to pass through the pores of the applicator by capillary action. The specific property of the gel form of the cosmetic composition is characterized by its “gel crossover point” which must be within a range that is suitable to allow passage through the pores of the porous applicator while maintain suspension of the powders and stable solubility of the salicylic acid.

In accordance with the exemplified embodiments, the cosmetic composition demonstrates effective reduction and prevention of oil and shine, control and reduction of acne and breakouts, which is delivered with a refreshing, cool skin feel, and is gentle on skin and does not disturb makeup.

Cosmetic Composition

According to the present invention, the cosmetic composition comprises, in various embodiments: (a) at least one beta hydroxy acid in a (b) hydroalcoholic gel dispersion, the hydroalcoholic gel dispersion comprising at least one each of (i) a C2-C5 alcohol, (ii) a powder, (iii) a polymer. The cosmetic composition may be dispensed using a porous applicator and is applied as an on-the-go skin refresher that is especially good for acne-prone skin. The cosmetic composition can be used throughout the day to control shine and minimize skin imperfections and discourage acne and is suitable for use alone or over or under make up.

According to the present invention, the cosmetic composition comprises, in some particular embodiments, a hydroalcoholic gel dispersion, comprising (a) at least one beta hydroxy acid selected from -n-octanoylsalicylic acid, salicylate, sodium salicylate, willow extract, beta hydroxybutanoic acid, tropic acid, and trethocanic acid, the beta hydroxy acid present in the cosmetic composition in an amount that is up to about 2.0%; (b) a hydroalcoholic gel, comprising: (i) at least one C2-C5 alcohol, and (ii) at least one polymer that includes acryloyldimethyltaurate/steareth-25 methacrylate crosspolymer; and (c) at least one powder that includes silica silylate, and optionally one or more additional powders selected from inorganic powders, organic powders, and silica; wherein the at least one beta hydroxy acid is dissolved in the hydroalcoholic gel and the at least one powder is dispersed in the hydroalcoholic gel.

In some embodiments, the cosmetic composition is free of, or substantially free of, or devoid of one or more of surfactants, oils, silicones, and film formers.

In some embodiments, the cosmetic composition has a gel crossover point ranging from about 5% to about 85% strain. In some embodiments from about 20% to about 80% strain, and in some embodiments from about 35% to about 75% strain, including all ranges and subranges there between.

In accordance with the various embodiments, the cosmetic composition is suitable for application to keratinous tissue any one or more of directly, under make up, and over make up. For purposes hereof, a “make up” as referred to herein can be any cosmetic composition utilized for application to a keratinous tissue for conferring at least an aesthetic effect, and in some examples containing a coloring agent which can provide color to a keratinous material to which it has been applied.

Beta Hydroxy Acid Component

In accordance with the various embodiments, the cosmetic composition comprises in the hydroalcoholic gel dispersion a component that comprises at least one beta hydroxy acid.

In some particular embodiments according to the disclosure, the composition includes salicylic acid present in the cosmetic composition in an amount that ranges from about 0.2% to about 2% by weight, based on the total weight of the composition.

The total amount of beta hydroxy acid includes in some embodiments from about 0.2% up to and not more than about 2% of beta hydroxy acid, by weight, based on the total weight of the cosmetic composition. In some embodiments, the cosmetic composition includes and up to and not more than about 1.9% of beta hydroxy acid. In some embodiments, the cosmetic composition includes from about 0.2% to about 1% of beta hydroxy acid. In some embodiments, the cosmetic composition includes and up to and not more than about 1% of beta hydroxy acid.

The term “beta-hydroxy acid” is understood to mean, according to the present invention, a carboxylic acid having a hydroxyl functional group and a carboxylic functional group separated by two carbon atoms. A beta hydroxy acid can be present in the cosmetic composition in the form of the free acid and/or in the form of one of its associated salts (salts with an organic base or an alkali metal, in particular), especially according to the final pH imposed on the composition.

The beta hydroxy acid is selected from one or more of salicylic acid and derivatives thereof (including 5-n-octanoylsalicylic acid, salicylate, sodium salicylate, and willow extract), beta hydroxybutanoic acid, tropic acid, and trethocanic acid.

In some embodiments, the composition includes a beta hydroxy acid comprising salicylic acid.

In accordance with the various embodiments, the amount of beta-hydroxy acid present in the compositions is in the range from about 0.2% to about 2%, and in some embodiments from about 0.2% to about 1.5%, and in some embodiments from about 0.2% to about 1%, including increments and all ranges and subranges therein and there between, by weight, based on the total weight of the composition. One of ordinary skill in the art, however, will appreciate that other ranges are within the scope of the invention.

Thus, the at least one beta hydroxy acid is present in the cosmetic composition from about 0.2, 0.3, 0.4, 0.5, 0.6, 0.7, 0.8, 0.9, 1.0, 1.2, 1.3, 1.4, 1.5, 1.6, 1.7, 1.8, 1.9, to about 2.0 weight percent, including increments and all ranges and subranges therein and there between.

Alcohol Component

In accordance with the various embodiments, the cosmetic composition comprises in the hydroalcoholic gel dispersion a component that comprises at least one alcohol. In addition, in some embodiments, the cosmetic composition may comprise at least one cooling component.

C2-C5 Alcohol and Water (Hydro-Alcoholic Solvent Component)

In some embodiments, the cosmetic composition comprises at least one C2-C5 alcohol. Suitable C2-C5 alcohols include ethanol, propanol, butanol, pentanol, isopropanol, isobutanol and isopentanol. In some embodiments the at least one alcohol is denatured. In some particular embodiments, the at least one alcohol comprises ethanol.

In some particular embodiments according to the disclosure, the composition includes alcohol, for example, denatured alcohol, present in the cosmetic composition in an amount that ranges from about 5% to about 20% by weight, based on the total weight of the composition. In some particular embodiments, denatured alcohol is present in the cosmetic composition in an amount that is about 20% by weight.

In some embodiments, the at least one alcohol is/are present in the cosmetic composition in amounts ranging from about 5% to about 30%, and in some embodiments from about 5% to about 25%, and in some embodiments from about 5% to about 20%, and in some embodiments from about 10% to about 15%, including increments and all ranges and subranges therein and there between, by weight, based on the total weight of the composition. One of ordinary skill in the art, however, will appreciate that other ranges are within the scope of the invention. Thus, the least one alcohol is present in the cosmetic composition from about 5, 6, 7, 8, 9, 10, 11, 12, 13, 14, 15, 1, 17, 18, 19, 20, 25, to about 30 weight percent, including increments and all ranges and subranges therein and there between.

In accordance with the various embodiments, the cosmetic composition includes water.

In various embodiments, the cosmetic composition comprises from about 5% to about 90% water, and in some embodiments from about 20% to about 85% water, and in some embodiments from about 50% to about 85% water, and in some embodiments from about 60% to about 80% water, and in some embodiments from about 65% to about 75%, water, including increments and all ranges and subranges therein and there between, by weight, based on the total weight of the composition. One of ordinary skill in the art, however, will appreciate that other ranges are within the scope of the invention. Thus, water is present, by weight, based on the total weight of the composition, from about 5, 10, 15, 20, 25, 30, 35, 40, 45, 50, 55, 60, 65, 66, 67, 68, 69, 70, 71, 72, 73, 74, 75, 80, 85, to about 90 weight percent, including increments and all ranges and subranges therein and there between.

The water used in the cosmetic composition may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

The pH of the cosmetic composition is in the range from about 1.0 to about 5.0, and in some embodiments, is in the range from about 3.5 to about 4.5, and in some embodiments in the range from about 3.9 and 4.5, and in some embodiments in the range from about 4.1 to about 4.4, and in some embodiments the pH is about 4.2. Generally, the pH can be adjusted to the desired value by addition of a base or an acid (organic or inorganic). In view of the inclusion of hydroxy acids in the cosmetic composition hereof, the pH is typically adjusted using a base, which in some examples is sodium hydroxide, or using an acid, which in some examples is citric acid.

Optional Cooling Component

Methyl Diisopropyl Propionamide

In some embodiments, the cosmetic composition may comprise one or more of menthol and methyl diisopropyl propionamide. In accordance with the various embodiments, methyl diisopropyl propionamide may be present in the cosmetic composition in an amount sufficient to provide a cooling effect to the keratinous material to which the cosmetic composition is applied. Whether a cooling effect is provided or not is a binary issue which is readily discernible by the user of the cosmetic composition based on typical experience with cooling sensations. In some embodiments, the cooling effect provided by methyl diisopropyl propionamide remains for at least 1 minute after application of the cosmetic composition, in some embodiments at least 2 minutes, in some embodiments at least 3 minutes, and in some embodiments at least 5 minutes, and can last as long as or more than 10 minutes after application.

In some embodiments, one of menthol and methyl diisopropyl propionamide may be present in the cosmetic composition in an amount ranging from about 0.05% to about 3% by weight, in some embodiments from about 0.075% to about 1% by weight, and in some embodiments from about 0.1% to about 0.5%, including increments and all ranges and subranges therein and there between, by weight, based on the total weight of the composition. One of ordinary skill in the art, however, will appreciate that other ranges are within the scope of the invention. Thus, each of one or more of menthol and methyl diisopropyl propionamide may be present in the cosmetic composition, by weight, based on the total weight of the composition, from about 0.05. 0.1, 0.15, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.5, to about 3.0 weight percent, including increments and all ranges and subranges therein and there between.

Powder Component

In accordance with the various embodiments, the cosmetic composition comprises in the hydroalcoholic gel dispersion a component that comprises at least one powder. In some embodiments, the cosmetic composition comprises at least two powders, and in some embodiments the cosmetic composition comprises three or more powders.

In some embodiments, the cosmetic composition comprises at least one inorganic powder and at least one organic powder. In some particular embodiments, the cosmetic composition comprises at least one inorganic powder and at least one aerogel. In some particular embodiments, the cosmetic composition comprises at least one inorganic powder, at least one organic powder, and at least one aerogel. In some particular embodiments, the cosmetic composition comprises powders that include perlite, silica silylate, and microcrystalline cellulose. In some particular embodiments, the cosmetic composition comprises powders that include perlite, silica silylate, silica and microcrystalline cellulose.

In some particular embodiments, the cosmetic composition excludes one or more powders comprising Nylon-12, polymethyl methacrylate, polyethylene beads, and powders which are considered microplastics.

In some embodiments, at least two powders are selected wherein the powders vary in particulate size.

In some particular embodiments according to the disclosure, the composition includes at least one inorganic powder and at least one organic powder and at least one aerogel. According to such embodiments, each of the at least one inorganic powder is present in the cosmetic composition in an amount that ranges from about 0.5% to about 1% by weight, based on the total weight of the composition. In some particular embodiments, the inorganic powder is present in the cosmetic composition in an amount that is about 0.8% by weight. Further according to such embodiments, each one of the at least one organic powder is present in the cosmetic composition in an amount that ranges from about 1.4% to about 2% by weight, based on the total weight of the composition. In some particular embodiments, the at least one organic powder is present in the cosmetic composition in an amount that is about 1.6% by weight. Further according to such embodiments, each one of the at least one aerogel is present in the cosmetic composition in an amount that ranges from about 0.2% to about 0.3% by weight, based on the total weight of the composition. In some particular embodiments, the at least one aerogel is present in the cosmetic composition in an amount that is about 0.25% by weight. According to some such embodiments, the powders include perlite, microcrystalline cellular, and a silica aerogel.

“Powder” should be understood as meaning solid particles which are insoluble in the medium of the cosmetic composition, whatever the temperature at which the cosmetic composition is manufactured. The powder component includes an oil absorbing powder.

In the various embodiments, the at least one powder is selected from particles of mineral, vegetable or organic origin.

The powders can be colorless or white and inorganic or organic, of any physical shape (platelet, spherical or oblong) and of any crystallographic form (for example sheet, cubic, hexagonal, orthorhombic and the like). The powders can be porous or nonporous.

In some embodiments, powders are selected from inorganic materials such as perlite; poly(vinyl chloride) powder; pumice (INCI name) such as pumice 3/B from Eyraud; ground fruit kernel shells, such as ground materials derived from apricot kernels or walnut shells; sawdust, wood flour or cork flour; glass beads; alumina (aluminium oxide) (INCI name: Alumina), such as the product sold under the name Dermagrain 900 by Marketech International; sugar crystal.

In some embodiments, the at least one powder is selected from particles comprising microcrystalline cellulose.

In some embodiments, the at least one powder is selected from silicas. In some embodiments, silicas that can be used may be natural and untreated. Mention may thus be made of the silicas provided under the names Sillitin N85, Sillitin N87, Sillitin N82, Sillitin V85 and Sillitin V88 by Hoffmann Mineral. Silicas may be fumed silicas. Silicas may be selected from silica powders under the name Silica Beads SB-700 by Miyoshi; Sunsphere® H51, Sunsphere® H33 by Asahi Glass. The silica particles different than the hydrophobic silica aerogel

In some embodiments, the at least one powder is selected from particles based on mannitol and cellulose which are sold under the Unisphere names by Induchem, agar-based capsules which are sold under the Primasponge names by Cognis and spheres based on jojoba esters which are sold under the Florasphères names by Floratech; and their mixtures. I

In some embodiments, the at least one powder is selected from particles formed of natural organic materials, such as starch powders, in particular powders formed of maize, wheat or rice starch, which are or are not crosslinked, such as powders formed of starch crosslinked with octenyl succinic anhydride, sold under the Dry-Flo name by National Starch.

Silica Aerogels

In some embodiments, the at least one powder is selected from one or more silica aerogel. Silica aerogels are porous materials obtained by replacing (by drying) the liquid component of a silica gel with air. They are generally synthesized via a sol-gel process in liquid medium and then dried, usually by extraction of a supercritical fluid, the one most commonly used being supercritical C02. This type of drying makes it possible to avoid shrinkage of the pores and of the material. The sol-gel process and the various drying processes are described in detail in Brinker C J., and Scherer G. W., Sol-Gel Science: New York: Academic Press, 1990. Silica aerogels, in general, have been disclosed in U.S. Pat. No. 9,320,689, the entire contents of which is hereby incorporated by reference.

In some embodiments, the aerogels that may be used according to the present invention are hydrophobic silica aerogels. The term “hydrophobic silica” means any silica whose surface is treated with silylating agents, for example halogenated silanes such as alkyl chlorosilanes, siloxanes, in particular dimethyl siloxanes such as hexamethyldisiloxane, or silazanes, so as to functionalized the OH groups with silyl groups Si—Rn, for example trimethylsilyl groups. Hydrophobic silica aerogels particles surface-modified with trimethylsilyl groups (trimethylsilyl silica) may be selected. As regards the preparation of hydrophobic silica aerogels particles that have been surface-modified by silylation, reference may be made to document U.S. Pat. No. 7,470,725, the entire contents of which is hereby incorporated by reference.

Suitable examples of hydrophobic silica aerogels that may be used In accordance with the various embodiments include the aerogel sold under the name VM-2260 (INCI name: Silica silylate), by the company Dow Corning, the particles of which have a mean size of about 1000 microns and a specific surface area per unit of mass ranging from 600 to 800 m2/g.

Mention may also be made of the aerogels sold by the company Cabot under the references AEROGEL TLD 201, AEROGEL OGD 201, AEROGEL TLD 203, ENOVA® AEROGEL MT 1100, ENOVA AEROGEL MT 1200.

In particular, the aerogel sold under the name VM-2270 (INCI name: Silica silylate), by the company Dow Corning, the particles of which have a mean size ranging from 5-15 microns and a specific surface area per unit of mass ranging from 600 to 800 m2/g, can be used.

In some embodiments, each one of the at least one powder is present in the cosmetic composition in amounts ranging from about 0.005% to about 10%, in some embodiments from about 0.05% to about 7.5%, in some embodiments from about 0.5% to about 5%, by weight, and in some embodiments from about 0.75% to about 2%, including increments and all ranges and subranges therein and there between, by weight, based on the total weight of the composition. One of ordinary skill in the art, however, will appreciate that other ranges are within the scope of the invention. Thus, each one of the at least one powder is present in the cosmetic composition from about 0.005, 0.01, 0.05, 0.1, 0.2, 0.3, 0.4, 0.5, 1, 1.5, 2, 2.5, 3, 3.5, 4, 4.5, 5, 6, 7, 8, 9, to about 10 weight percent, including increments and all ranges and subranges therein and there between.

In some embodiments, each one of the at least one powder comprising silica aerogel is present in the cosmetic composition in amounts ranging from about 0.05% to about 10%, in some embodiments from 0.075% to 5%, and in some embodiments from 0.1% to 0.5%, including increments and all ranges and subranges therein and there between, by weight, based on the total weight of the composition. One of ordinary skill in the art, however, will appreciate that other ranges are within the scope of the invention. Thus, each one of the at least one powder comprising silica aerogel is present in the cosmetic composition from about 0.05. 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.2, 1.3, 1.4, 1.5, 1.6, 1.7, 1.8, 1.9, 2.0, 2.5, 3.0, 3.5, 4.0, 4.5, to about 3.0 weight percent, including increments and all ranges and subranges therein and there between.

In some embodiments, the total combined amount of powder(s) including silica aerogel(s) present in the cosmetic composition is less than 12% by weight with respect to the total weight of the cosmetic composition, including in the range from about 0.5% to about 10%, in some embodiments from about 1% to 8%, in some embodiments from about 1% to about 3%, and in some embodiments from about 1.5% to about 2.5%, including increments and all ranges and subranges therein and there between, by weight, based on the total weight of the composition. One of ordinary skill in the art, however, will appreciate that other ranges are within the scope of the invention. Thus, the total combined amount of powder(s) including silica aerogel(s) present in the cosmetic composition is present from about 0.5, 0.6, 0.7, 0.8, 0.9, 1.0, 2, 3, 4, 5, 6, 7, 8, 9, 10, 11, to about 12 weight percent, including increments and all ranges and subranges therein and there between.

Polymer Component

In accordance with the various embodiments, the cosmetic composition comprises in the hydroalcoholic gel dispersion a component that comprises at least one polymer. In the various embodiments the at least one polymer is not electrolyte sensitive or salt intolerant, such that the at least on polymer is suitable to maintain suspension/dispersion of the at least one powder at low pH and in the presence of salts/electrolytes. Polymers that are electrolyte sensitive/salt intolerant with either (1) not fully swell in water or other solvents or (2) collapse/break in the presence of electrolytes. This will result in a low/no viscosity gel that is incapable of suspending the powders.

In some particular embodiments according to the disclosure, the composition includes at least one amphiphilic polymer present in the cosmetic composition in an amount that ranges from about 0.9% to about 2.4% by weight, based on the total weight of the composition. In some particular embodiments, the at least one amphiphilic polymer is present in the cosmetic composition in an amount that is about 1.13% by weight. According to some such embodiments, the at least one amphiphilic polymer is Acryloyldimethyltaurate/Steareth-25 Methacrylate Crosspolymer (Aristoflex HMS) (ethoxylated copolymer of AMPS/stearyl methacrylate crosslinked with trimethylol triacrylate).

Amphiphilic Polymers

In some embodiments, the cosmetic composition comprises at least one polymer selected from amphiphilic polymers that include, but are not limited to, hydrophobically-modified sulfonic acid copolymers such as Ammonium Acryloyldimethyltaurate/Steareth-25 Methacrylate Crosspolymer (Aristoflex HMS) (ethoxylated copolymer of AMPS/stearyl methacrylate crosslinked with trimethylol triacrylate), Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant) (crosslinked ethoxylated AMPS/behenyl methacrylate), Aristoflex SNC (crosslinked ethoxylated AMPS/C16-C18), Aristoflex LNC (noncrosslinked AMPS/C12-C14), and mixtures thereof.

In some particular embodiments, the cosmetic composition comprises Ammonium Acryloyldimethyltaurate/Steareth-25 Methacrylate Crosspolymer (Aristoflex HMS) (ethoxylated copolymer of AMPS/stearyl methacrylate crosslinked with trimethylol triacrylate).

In some embodiments, the cosmetic composition may comprise a polymer present from greater than about 1% by weight based on the weight of the composition, and selected from polyacrylate crosspolymer 6 (Sepimax Zen), and an acrylates/C10-30 Alkyl acrylate crosspolymer (Ultrez 20 or 21).

In some embodiments, the cosmetic composition excludes a polyacrylate polymer, for example, the cosmetic composition excludes at least one or all of polyacrylate crosspolymer 6, and an acrylates/C10-30 Alkyl acrylate crosspolymer.

In some embodiments, the cosmetic composition may comprise at least one amphiphilic polymer. In some embodiments, the amphiphilic polymer comprises at least one ethylenically unsaturated monomer containing a sulphonic group, in freeform or partially or totally neutralized form and comprising at least one hydrophobic portion. The hydrophobic portion present in these polymers in some embodiments contains from 6 to 50 carbon atoms, in some embodiments from 6 to 22 carbon atoms, in some embodiments from 6 to 18 carbon atoms and in some embodiments from 12 to 18 carbon atoms, including all ranges and subranges there between.

The amphiphilic polymers may be based on at least one ethylenically unsaturated hydrophilic monomer A and on at least one hydrophobic monomer B. In some embodiments, the monomer A comprises a strong acid function, in particular a sulphonic acid or phosphonic acid function. The hydrophobic monomer B comprises at least one hydrophobic radical, chosen from: saturated or unsaturated C6-C18 linear alkyl radicals (for example, n-hexyl, n-octyl, n-decyl, n-hexadecyl, n-dodecyl or oleyl); branched alkyl radicals (for example, isostearic) or cyclic alkyl radicals (for example, cyclododecane or adamantane); C6-C18 fluoro or alkylfluoro radicals (for example, the group of formula —(CH2)2-(CF2)9-CF3); a cholesteryl radical or radicals derived from cholesterol (for example, cholesteryl hexanoate); aromatic polycyclic groups, for instance naphthalene or pyrene; and silicone or alkyl silicone or alkylfluorosilicone radicals. Among these radicals, linear and branched alkyl radicals may be selected.

The amphiphilic polymers are in some embodiments water-soluble or water-dispersible in neutralized form. Their viscosity at a concentration of 1% in water, at a shear rate of 1 s-1, at a pH of from about 5 and 8, at 25° C., is in some embodiments less than 5 000 mPa·s. Amphiphilic polymers suitable for use in the present invention are described in US 2001/049419 and U.S. Pat. No. 6,645,476, the entire contents of both of which are hereby incorporated by reference.

The amphiphilic polymers may be crosslinked. The crosslinking agents may be chosen from, for example, the polyolefinically unsaturated compounds commonly used for crosslinking polymers obtained by free-radical polymerization. According to one embodiment of the invention, the crosslinking agent is chosen from methylenebisacrylamide, allyl methacrylate or trimethylolpropane triacrylate (TMPTA). The degree of crosslinking, in some embodiments, ranges from 0.01 mol % to 10 mol %, and in some embodiments from 0.2 mol % to 2 mol %, relative to the polymer, including all ranges and subranges there between.

In some embodiments, amphiphilic polymers may be chosen from crosslinked or non-crosslinked amphiphilic polymers of 2acrylamido-2-methylpropanesulphonic (AMPS) acid and of at least one ethylenically unsaturated monomer comprising at least one hydrophobic portion containing from 6 to 30 carbon atoms, in some embodiments from 6 to 22 carbon atoms, in some embodiments from 6 to 18 carbon atoms and in some embodiments from 12 to 18 carbon atoms, including all ranges and subranges there between.

The amphiphilic polymers may be partially or totally neutralized with a mineral base (for example, sodium hydroxide, potassium hydroxide or aqueous ammonia) or an organic base such as monoethanolamine, diethanolamine, triethanolamine, aminomethylpropanediol, N-methylglucamine, or basic amino acids, for instance arginine and lysine, and mixtures thereof.

Suitable examples of amphiphilic polymers include, but are not limited to, hydrophobically-modified sulfonic acid copolymers such as Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant) (crosslinked ethoxylated AMPS/behenyl methacrylate), Ammonium Acryloyldimethyltaurate/Steareth-25 Methacrylate Crosspolymer (Aristoflex HMS) (ethoxylated copolymer of AMPS/stearyl methacrylate crosslinked with trimethylol triacrylate), Aristoflex SNC (crosslinked ethoxylated AMPS/C16-C18), Aristoflex LNC (noncrosslinked AMPS/C12-C14), and mixtures thereof.

In some embodiments, the at least one polymer is present in the cosmetic composition in amounts ranging from about 0.05% to about 5%, in some embodiments from about 0.1% to about 1%, and in some embodiments from about 0.2% to about 0.5%, including increments and all ranges and subranges therein and there between, by weight, based on the total weight of the composition. One of ordinary skill in the art, however, will appreciate that other ranges are within the scope of the invention. Thus, the at least one polymer is present in the cosmetic composition from about 0.05. 0.1, 0.15, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.5, 3.0, 3.5, 4.0, 4.5 to about 5.0 weight percent, including increments and all ranges and subranges therein and there between.

Optional Additives

The cosmetic composition may also include one or more additives used in the cosmetics field which do not affect the properties of the cosmetic composition according to the invention, such as one or more of any of an antioxidant, vitamin, essential oil, botanical extract, pH adjuster/neutralizing agent, fragrance, odor absorber, preservative, an anti-microbial, including, but not limited to, phenoxyethanol, chlorphenesin, capryloyl glycol, and combinations thereof. In some embodiments, the cosmetic composition includes additives selected from fragrance, and phenoxy ethanol.

Although the optional additives are given as examples, it will be appreciated that other optional components compatible with cosmetic applications known in the art may be used.

In accordance with the various embodiments, the amount of each additive, when present in the cosmetic composition, can be present in a range from about 0.001% to about 20%, and in some embodiments from about 0.005% to about 0.01%, and in some embodiments from about 0.01% to about 0.1%, and in some embodiments from about 0.15% to about 5%, and in some embodiments from about 0.40% to about 4%, and in some embodiments from about 0.5% to about 2.5%, and in some embodiments from about 1% to about 2%, including increments and all ranges and subranges therein and there between, by weight, based on the total weight of the composition. One of ordinary skill in the art, however, will appreciate that other ranges are within the scope of the invention. And in some embodiments, a combination of additives present in the cosmetic composition can be present in a range from about 0.001% to about 20%. Thus, any one or a combination of additives may be present in the cosmetic composition from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 weight percent, including increments and all ranges and subranges therein and there between.

Cosmetic Dispensing System

In some embodiments of the cosmetic composition according to the disclosure, the cosmetic composition may, as desired, be selected for use with a cosmetic dispensing system that includes a porous applicator. Of course, it will be appreciated that while some embodiments of the cosmetic composition may be suited for use with a porous applicator as described herein, such use is not a required aspect of the instant invention. Thus, it will be further appreciated that embodiments of the cosmetic composition may not be suited for use with the applicator. Thus, according to the present invention, the cosmetic composition hereof may be selected for use as a component of a cosmetic dispensing system which comprises, in various embodiments, a cosmetic composition that has a gel crossover point suitable for dispensing through a porous applicator, and a dispenser that includes a container that includes a retainer and an absorbent component disposed within the retainer, and a porous applicator comprising a porous material that is in contact on a first side with the absorbent component in the retainer, and is exposed on a second side for contacting keratinous tissue to be treated. The dispenser may further comprise a cap or lid for covering the porous applicator when the dispensing system is not in use. The dispenser may have any shape or size, and thus may be provided in a container design that is similar to any of a variety of cosmetic applicators having shapes that are cylindrical, hemispherical or spherical, block shaped, tubular, pencil shaped, and the like. It will be appreciated that the shape is not limiting. And the material forming each of the container, including the retainer, the absorbent component, and the porous applicator may be formed of any one or combination of plastic, natural fibers, synthetic fibers, ceramic, metal, natural sponge or other nature-based material and the like.

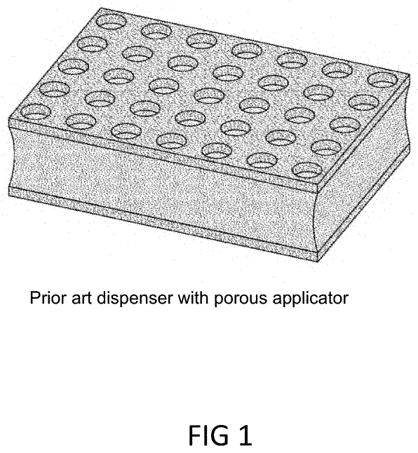

Reference may be made to PCT application WO2018152472A1 which discloses a liquid applicator for applying a liquid or gel to a surface. The liquid applicator comprises a sintered porous elastomeric material body. Liquid moves from one end of the applicator through to another end of the liquid applicator by pushing the liquid through the applicator. The sintered porous elastomeric body can be selected from the group consisting of ethylene vinyl acetate, polypropylene, polyethylene for example high density polyethylene, low density polyethylene or ultrahigh molecular weight polyethylene and has an average pore size greater than 20 microns, greater than 40 microns, greater than 60 microns, greater than 80 microns, greater than 100 microns, greater than 125 microns, greater than 150 microns, greater than 175 microns, greater than 200 microns, or greater than 250 microns. In some embodiments, the sintered porous elastomeric body has an average pore size less than about 300 microns. Referring to the instant drawings, FIG. 1 shows a representative embodiment of a dispenser that is disclosed in the above identified PCT application as FIG. 23.

The cosmetic composition is absorbed and retained in the retainer, which includes in some embodiments a sponge, and retains the cosmetic composition in a dispersed gel state until dispensed for application to skin. The dispenser is actuated to dispense the cosmetic composition onto skin by transfer first from the sponge to the porous applicator and then passage through the porous applicator toward the outer surface by capillary action. In some embodiments, the actuation may be achieved by compression of the exposed side of the porous applicator. According to the process of dispensing, the cosmetic composition must convert from a gel to a liquid in order to pass through the pores of the applicator by capillary action. The specific property of the gel form of the cosmetic composition is characterized by its gel crossover point which must be within a range that is suitable to allow passage through the pores of the porous applicator while maintain suspension of the powders and stable solubility of the salicylic acid.

In embodiments suitable for use with the cosmetic dispensing system, the cosmetic composition has a gel crossover point ranging from about 5% to about 85% strain. In some embodiments from about 20% to about 85% strain, in some embodiments from about 25% to about 80%, and in some embodiments from about 35% to about 75% strain, including all ranges and subranges there between. In the various embodiments of the cosmetic composition that is formulated for delivery using a porous applicator has a gel crossover point which allows it to suspend the at least one powder and pass through a porous applicator with shear. Thus, in some embodiments, of the cosmetic composition that is formulated for delivery using a porous applicator, the cosmetic composition is formulated to have a gel crossover point that is suited to the pore size of an applicator.

In embodiments suitable for use with the cosmetic dispensing system, the porous applicator of the cosmetic dispensing system includes on at least the exposed surface intended for contacting skin, a plurality of pores having an average size in a range from 1 to about 1000 microns, and in some embodiments, in a range from about 10 to about 50 microns, and in some embodiments in a range from about 25 microns to about 250 microns, and in some embodiments in a range from about 30 to about 100 microns. In some embodiments, the porous applicator of the cosmetic dispensing system includes, on at least the first surface intended for contacting the sponge containing the cosmetic composition in the retainer, a plurality of pores having an average size in a range from 1 to about 1000 microns, and in some embodiments, in a range from about 10 to about 50 microns, and in some embodiments in a range from about 25 microns to about 250 microns, and in some embodiments in a range from about 30 to about 100 microns., and the portion of the porous material at the bottom of the base portion of the container has an average pore size of about 90 to 200 microns. In some embodiments, the porous applicator of the cosmetic dispensing system includes from the first surface to the second surface throughout the porous material, a plurality of pores having an average size in a range from 1 to about 1000 microns, and in some embodiments, in a range from about 10 to about 50 microns, and in some embodiments in a range from about 25 microns to about 250 microns, and in some embodiments in a range from about 30 to about 100 microns., and the portion of the porous material at the bottom of the base portion of the container has an average pore size of about 90 to 200 microns.

Thus, in various embodiments, pores on any one surface or through the entire material from the first surface to the second surface have a pore size in the range from about 1, 2, 3, 4, 5, 6, 7, 8, 9, 10, 20, 30, 40, 50, 60, 70 80, 90, 100, 200, 300, 400, 500, 600, 700, 800, 900 to about 1000 microns, including increments and all ranges and subranges therein and there between According to some embodiments, the pores are interconnecting. According to the various embodiments, the porosity through the material from the first surface to the second surface is one of uniform and continuous, variable from larger pores at the first surface to smaller pores at the second surface, and variable from smaller pores at the first surface to larger pores at the second surface.

The porous material may be formed of any suitable material that may be one or a combination of gel-foam, polymeric, ceramic, metal and the like.

Methods for Enhancing the Appearance of Acne Prone Skin

According to the present invention, the method for enhancing the appearance of acne prone skin comprises, in various embodiments, includes providing a dispensing system as disclosed herein wherein the dispenser containing the cosmetic composition is used to apply the composition to skin that is prone to acne. The dispenser system may be utilized to apply the cosmetic composition by stroking using a dabbing motion to dispense a light coating of the cosmetic composition onto select areas of the skin in dispensed doses that are in an amount that is from about 0.01 mg to about 100 mg of the cosmetic composition, and in discrete doses per dab in a range from about 0.01 mg to about 100 mg/dab, or from about 0.25 mg to about 25 mg per dab, or from about 0.5 mg to about 20 mg/dab, or from about 1 mg to about 15 mg/dab, or from about 2 mg to about 10 mg/dab. In some embodiments, a single use of the cosmetic composition will include application of about 1-10 dabs or about 2-6 dabs, and in some embodiments about 4 dabs. Thus, in some embodiments, a dose application of the cosmetic composition includes about 4 dabs at a concentration per dab in the range from about 0.25 mg-25 mg. In one example a dose application may be 1 mg up to about 100 mg, which may be delivered in a single application stroke or in more than one application stroke.

### EXAMPLES

### Example 1: Raw Materials

The raw materials as used in the inventive composition include Salicylic acid, perlite, microcrystalline cellulose, silica silylate, alcohol denatured, and acryloyldimethyltaurate/steareth-25 methacrylate crosspolymer.

### Example 2: Inventive Compositions

Exemplary embodiments of the inventive composition is set forth in Table 1A and Table 1B, below.

TABLE 1A
Inventive compositions

| INGREDIENT | INV 1 | INV 2 | INV 3 | INV 4 |
|---|---|---|---|---|
| WATER | 75.93 | 75.1 | 74.53 | 73.73 |
| AMMONIUM | 0.9 | 1.13 | 1.4 | 1.8 |
| ACRYLOYLDIMETHYL- | | | | |
| TAURATE/STEARETH- | | | | |
| 25 METHACRYLATE | | | | |
| CROSSPOLYMER | | | | |
| ALCOHOL DENAT. | 20 | 20 | 20 | 20 |
| SALICYLIC ACID | 0.2 | 0.5 | 1 | 1.5 |
| SODIUM HYDROXIDE | 0.12 | 0.12 | 0.22 | 0.12 |
| METHYL DIISOPROPYL | 0.2 | 0.2 | 0.2 | 0.2 |
| PROPIONAMIDE | | | | |
| PERLITE | 0.8 | 0.8 | 0.8 | 0.8 |
| MICROCRYSTALLINE | 1.6 | 1.6 | 1.6 | 1.6 |
| CELLULOSE | | | | |
| SILICA SILYLATE | 0.25 | 0.25 | 0.25 | 0.25 |
| SODIUM HYDROXIDE | 0 | 0 | 0 | 0 |
| CITRIC ACID | 0 | 0 | 0 | 0 |
| FRAGRANCE/PARFUM | | 0.3 | | |
| PHENOXYETHANOL | | | | |
| MENTHOL | | | | |
| Viscosity (M2, 24 h), uD | 16.9 | 20 | 22.1 | 31.3 |
| Surface tension, mN/m | | 26.23 | | |
| Crossover modulus, % | | 59.7 | | |

TABLE 1 B
Inventive Compositions continued

| INGREDIENT | INV 5 | INV 6 | INV 7 | INV 8 |
|---|---|---|---|---|
| WATER | 72.8 | 84.8 | 89.55 | 72.35 |
| AMMONIUM | 2.05 | 1.13 | 1.13 | 2.4 |
| ACRYLOYLDIMETHYL- | | | | |
| TAURATE/STEARETH- | | | | |
| 25 METHACRYLATE | | | | |
| CROSSPOLYMER | | | | |
| ALCOHOL DENAT. | 20 | 10 | 5 | 20 |
| SALICYLIC ACID | 1.9 | 0.5 | 0.5 | 2 |
| SODIUM HYDROXIDE | 0.4 | 0.12 | 0.12 | 0.4 |
| METHYL DIISOPROPYL | 0.2 | 0.2 | 0.2 | 0.2 |
| PROPIONAMIDE | | | | |
| PERLITE | 0.8 | 0.8 | 0.8 | 0.8 |
| MICROCRYSTALLINE | 1.6 | 1.6 | 1.6 | 1.6 |
| CELLULOSE | | | | |
| SILICA SILYLATE | 0.25 | 0.25 | 0.25 | 0.25 |
| SODIUM HYDROXIDE | 0 | 0 | 0 | 0 |
| CITRIC ACID | 0 | 0 | 0 | 0 |
| FRAGRANCE/PARFUM | | | | |
| PHENOXYETHANOL | | 0.6 | 0.6 | 0.001 |
| MENTHOL | | | 0.1 | |
| Viscosity (M2, 24 h), uD | | | 31.1 | |
| Surface tension, mN/m | | | 40.16 | |
| Crossover modulus, % | | | 79 | |

### Example 3: Comparative Compositions

Comparative composition are set forth in Table 2A and Table 2B, below.

TABLE 2A
Comparative Compositions

| INGREDIENT | COMP 1 | COMP 2 | COMP 3 |
|---|---|---|---|
| WATER | 69.96 | 76.33 | 73.21 |
| AMMONIUM | 0.35 | 0.5 | 2.25 |
| ACRYLOYLDIMETHYLTAURATE/STEARETH- | | | |
| 25 METHACRYLATE CROSSPOLYMER | | | |
| ALCOHOL DENAT. | 25 | 20 | 20 |
| METHYL DIISOPROPYL PROPIONAMIDE | 0.2 | 0.2 | |
| | 0.1 | | |
| TRIETHANOLAMINE | 0 | | |
| PERLITE | 0.63 | 0.8 | 0.6 |
| CELLULOSE | 2 | | |
| MICROCRYSTALLINE CELLULOSE | 1.5 | 1.6 | 1.2 |
| SILICA SILYLATE | 0.26 | 0.25 | 0.25 |
| SALICYLIC ACID | | 0.2 | 2 |
| SODIUM HYDROXIDE | | 0.12 | 0.49 |
| POLYACRYLATE CROSSPOLYMER-6 | | | |
| CHARCOAL POWDER (and) POLYGLYCERYL- | | | |
| 10 STEARATE (and) POLYGLYCERYL-10 | | | |
| MYRISTATE (and) POLYGLYCERIN-10 | | | |
| FRAGRANCE/PARFUM | | | |
| | No anti-acne | Addition of | 2% salicylic acid |
| | active (from | 0.2% salicylic | could not be |
| | US20190231663) | acid (anti-acne | stabilized with |
| | | active) to a | 2.25% polymer, |
| | | formula like | even with lower |
| | | COMP 1 | powder levels |
| | | induces | (separation into |
| | | instability | two distinct |
| | | (separation into | layers) |
| | | two distinct | |
| | | layers) | |

TABLE 2A
Comparative Compositions continued

| INGREDIENT | COMP 4 | COMP 5 |
|---|---|---|
| WATER | 75.06 | 77.75 |
| AMMONIUM | | 1.13 |
| ACRYLOYLDIMETHYL- | | |
| TAURATE/STEARETH- | | |
| 25 METHACRYLATE | | |
| CROSSPOLYMER | | |
| ALCOHOL DENAT. | 20 | 20 |
| METHYL DIISOPROPYL | | 0.2 |
| PROPIONAMIDE | | |
| TRIETHANOLAMINE | | |
| PERLITE | 0.6 | |
| CELLULOSE | | |
| MICROCRYSTALLINE | 1.2 | |
| CELLULOSE | | |
| SILICA SILYLATE | 0.25 | |
| SALICYLIC ACID | 2 | 0.5 |
| SODIUM HYDROXIDE | 0.49 | 0.12 |
| POLYACRYLATE | 0.4 | |
| CROSSPOLYMER-6 | | |
| CHARCOAL POWDER (and) | | |
| POLYGLYCERYL-10 STEARATE | | |
| (and) POLYGLYCERYL-10 | | |
| MYRISTATE (and) | | |
| POLYGLYCERIN-10 | | |
| FRAGRANCE/PARFUM | | 0.3 |
| | 0.25% salicylic | Inferior |
| | acid could not be | sweat |
| | stabilized with 0.4% | resistance |
| | of an alternative | |
| | polymer, even with | |
| | lower powder levels | |
| | (separation into two | |
| | distinct layers) | |

### Example 4. Evaluation of Salicylic Acid Solubility and Stability of Inventive Compositions

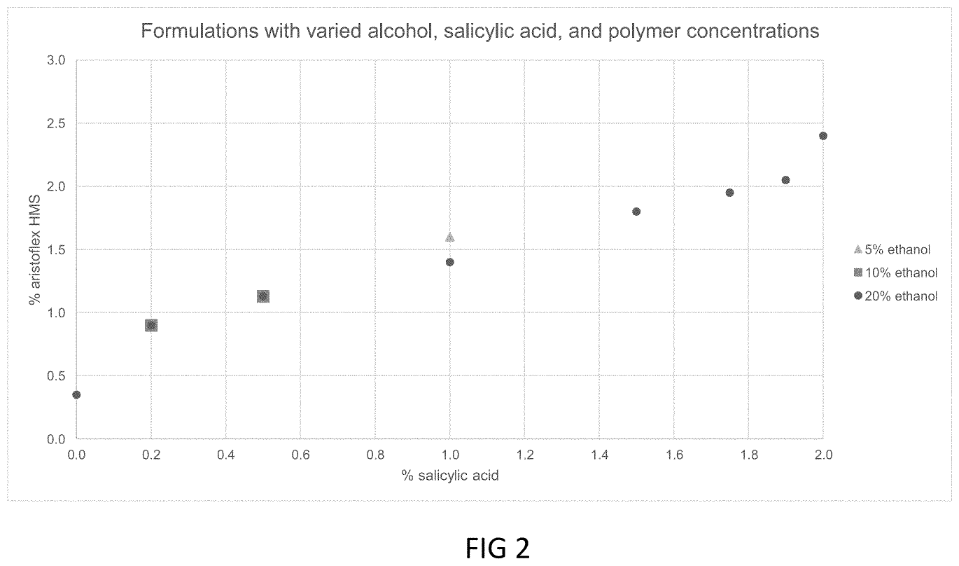

Compositions were evaluated for salicylic acid solubility and gel stability and the results are shown in FIG. 2 and Table 3. Stability of the cosmetic composition according to this invention was measured by visual and odor assessment, pH, and viscosity over a two-month period at the following temperatures: 4, 25, 37, and 45*C, as well as a 10-day freeze/thaw cycle.

TABLE 3
Formulas tested for composition stability

| FORMULA | Type of | INGREDIENT WT % | | | | | | | Stable |
|---|---|---|---|---|---|---|---|---|---|
| # | formula | ACID | PWDR | PWDR | PWDR | ALC | POLY 1 | POLY 2 | Y/N |
| 1 | inventive | 0.2 | 0.8 | 1.6 | 0.25 | 20 | 0.9 | | Y |
| 2 | inventive | 0.5 | 0.8 | 1.6 | 0.25 | 20 | 1.13 | | Y |
| 3 | inventive | 1 | 0.8 | 1.6 | 0.25 | 20 | 1.4 | | Y |
| 4 | inventive | 1.5 | 0.8 | 1.6 | 0.25 | 20 | 1.8 | | Y |
| 5 | inventive | 1.9 | 0.8 | 1.6 | 0.25 | 20 | 2.05 | | Y |
| 6 | inventive | 0.5 | 0.8 | 1.6 | 0.25 | 10 | 1.13 | | Y |
| 7 | inventive | 0.5 | 0.8 | 1.6 | 0.25 | 5 | 1.13 | | Y |
| 8 | inventive | 2 | 0.8 | 1.6 | 0.25 | 20 | 2.4 | | Y |
| COMP 1 | comparative | 0 | 0.8 | 1.6 | 0.25 | 20 | 0.35 | | Y |
| COMP 2 | comparative | 0.2 | 0.8 | 1.6 | 0.25 | 20 | 0.5 | | N |
| COMP 3 | comparative | 2 | 0.6 | 1.2 | 0.25 | 20 | 2.25 | | N |
| COMP 4 | comparative | 2 | 0.6 | 1.2 | 0.25 | 20 | | 0.4 | N |
| EXP 14 | Experimental | 0.2 | 0.8 | 1.6 | 0.25 | 20 | 0.7 | | N |
| EXP 15 | Experimental | 0.5 | 0.6 | 1.2 | 0.25 | 20 | 0.6 | | N |
| EXP 16 | Experimental | 0.5 | 0.6 | 1.2 | 0.25 | 20 | | 0.4 | N |
| EXP 17 | Experimental | 0.5 | 0.6 | 1.2 | 0.25 | 20 | | 0.75 | N |
| EXP 18 | Experimental | 0.5 | 0.6 | 1.2 | 0.25 | 20 | 0.9 | | N |
| EXP 19 | Experimental | 1 | 0.8 | 1.6 | 0.25 | 20 | 1.1 | | N |
| EXP 20 | Experimental | 1 | 0.8 | 1.6 | 0.25 | 20 | 1.2 | | N |
| EXP 21 | Experimental | 1 | 0.8 | 1.6 | 0.25 | 20 | 1.3 | | N |
| EXP 22 | Experimental | 1.5 | 0.8 | 1.6 | 0.25 | 20 | 1.6 | | N |
| EXP 23 | Experimental | 1.5 | 0.8 | 1.6 | 0.25 | 20 | 2 | | Y |
| EXP 24 | Experimental | 1.75 | 0.8 | 1.6 | 0.25 | 20 | 1.95 | | Y |
| EXP 25 | Experimental | 2 | 0.6 | 1.2 | 0.25 | 20 | 1.13 | | N |
| EXP 26 | Experimental | 2 | 0.8 | 1.6 | 0.25 | 20 | 1.13 | | N |
| EXP 27 | Experimental | 2 | 0.6 | 1.2 | 0.25 | 20 | 1.7 | | N |
| EXP 28 | Experimental | 2 | 0.6 | 1.2 | 0.25 | 20 | 1.8 | | N |
| EXP 29 | Experimental | 2 | 0.8 | 1.6 | 0.25 | 20 | 2.6 | | N |
| EXP 30 | Experimental | 2 | 0.6 | 1.2 | 0.25 | 20 | | 0.75 | N |
| EXP 31 | Experimental | 0.2 | 0.8 | 1.6 | 0.25 | 10 | 0.9 | | Y |
| EXP 32 | Experimental | 0.5 | 0.8 | 1.6 | 0.25 | 4 | 1.13 | | N |
| EXP 33 | Experimental | 0.5 | 0.8 | 1.6 | 0.25 | 10 | 1.3 | | Y |
| EXP 34 | Experimental | 0.5 | 0.8 | 1.6 | 0.25 | 15 | 1.13 | | Y |
| EXP 35 | Experimental | 1 | 0.8 | 1.6 | 0.25 | 5 | 1.4 | | N |
| EXP 36 | Experimental | 1 | 0.8 | 1.6 | 0.25 | 5 | 1.6 | | Y |
| EXP 37 | Experimental | 1 | 0.8 | 1.6 | 0.25 | 10 | 1.4 | | N |

Ingredient Key for Table 3: ACID: Salicylic acid; PWDR: Perlite; PWDR: Microcrystalline cellulose; PWDR: silica silylate; ALC: alcohol denat; POLY 1: acryloyldimethyltaurate/steareth-25 methacrylate crosspolymer; POLY 2: polyacrylate crosspolymer-6.

Referring to the drawings, FIG. 2 shows the effects of increasing polymer and alcohol on the solubility of salicylic acid. Further testing with various formulations, as shown in Table 3 below, demonstrate examples of inventive compositions in which the hydroalcoholic gel dispersion of powders is stabilized in the presence of salicylic acid.

Referring now to Table 3, each of the inventive compositions demonstrates stability as evidenced by no separation, where INV 3 is stable with 1% salicylic acid, and INV 6 is stable with 2% salicylic acid. Unstable prototypes typically exhibited a clear separation into two distinct layers or a significant change in viscosity. For example, the sample prepared using 2% salicylic acid and 2.25% acryloyldimethyltaurate/steareth-25 methacrylate crosspolymer (COMP 3) separated into two distinct layers over time. The addition of only 0.2% salicylic acid to 0.5% acryloyldimethyltaurate/steareth-25 methacrylate crosspolymer (COMP 2) caused the formula to separate into two layers, whereas (COMP 1) (a comparative composition herein and an inventive composition disclosed in US20190231663A1), is stable with only 0.35% acryloyldimethyltaurate/steareth-25 methacrylate crosspolymer but no salicylic acid. In contrast, the cosmetic composition according to this invention, shown using (INV 2) is stable with 0.5% salicylic acid and 1.13% acryloyldimethyltaurate/steareth-25 methacrylate crosspolymer.

### Example 5. Evaluation of Gel Crossover of Inventive and Comparative Compositions

TABLE 4
Gel crossover point determined using a Discovery
Hybrid rheometer by TA Instruments

| FORMULA # | GEL-SOL POINT, % |
|---|---|
| INV 2 | 59.7 |
| INV 7 | 79.0 |
| EXP 38 (INV 2 + 0.5% xanthan gum in place | 219.0 |
| of acryloyldimethyltaurate/steareth-25 | |
| methacrylate crosspolymer) | |

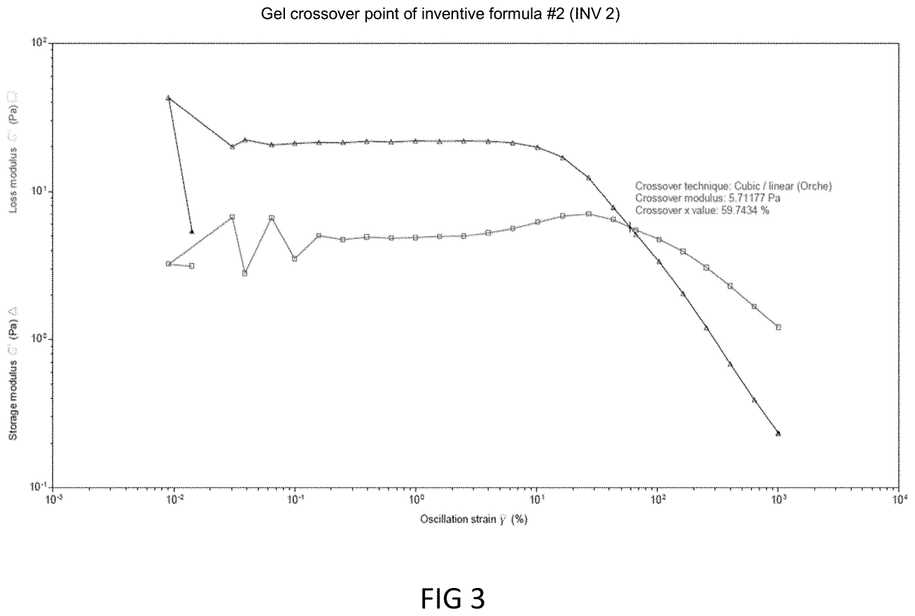

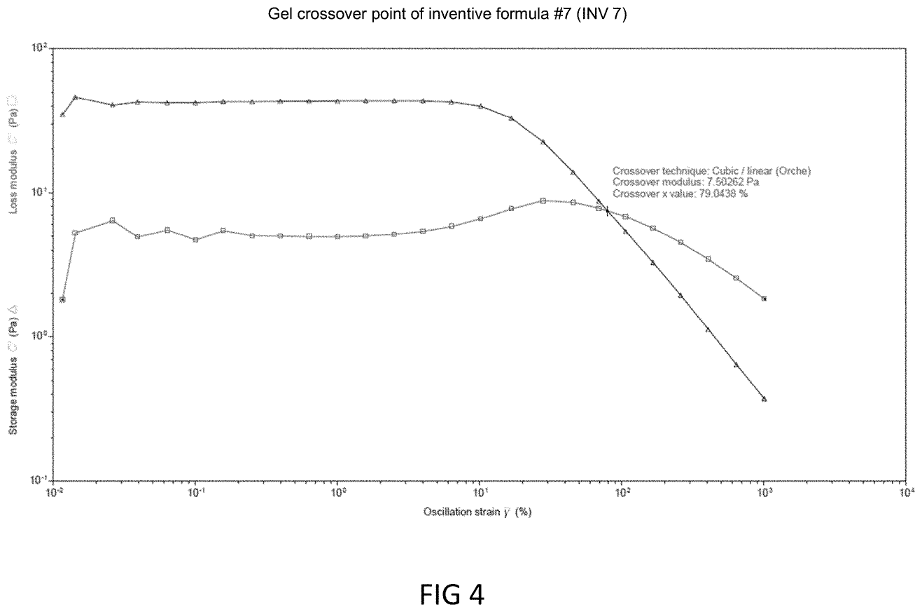

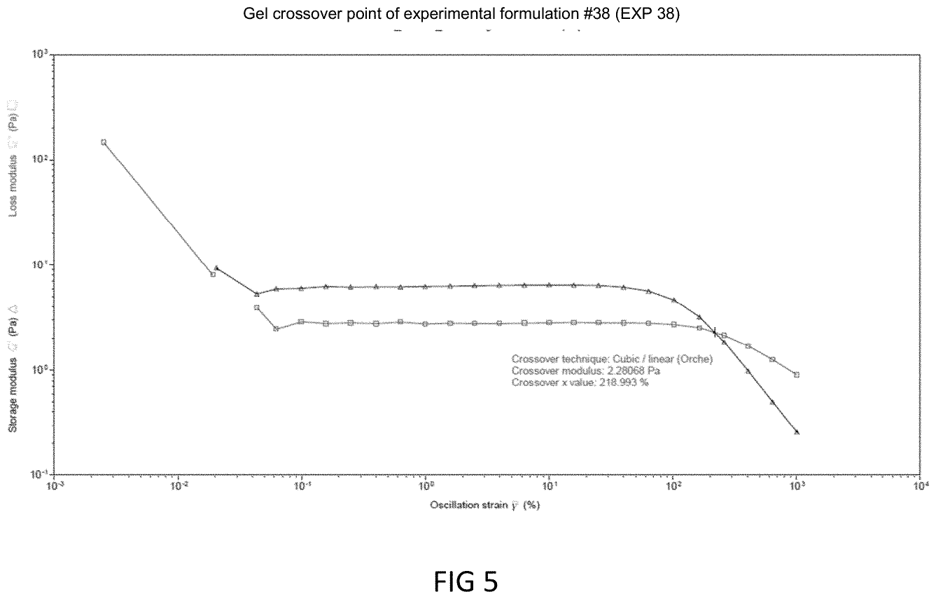

Compositions were evaluated for gel crossover point; representative examples are shown in FIG. 3-FIG. 5 and in Table 4. Gel Crossover Point” (Sol/Gel Point), means the point at which the G″ (loss modulus) intersects the G′ (storage modulus), and is determined, in one example, using a Discovery HR-2 Rheometer by TA Instruments having 40 mm parallel plate geometry on a stainless-steel flat Peltier plate. The test can be run @ 20° C., with test parameter of angular frequency of 1.0 rad/s and logarithmic sweep: Strain % 0.01 to 1000.0%. 5 points per decade. Results are reported in % strain.

Referring again to FIG. 3-FIG. 5 and Table 4, INV 2 is stable with 0.5% salicylic acid, has a gel crossover point of 59.7% strain allowing it to pass through a porous applicator, and, INV 7 is stable with 0.5% salicylic acid at a lower alcohol level than INV 2 (5% vs 20%) and has a gel crossover point of 79% strain allowing it to pass through a porous applicator. Notably, with reference to EXP 38, which is a variation of INV 2 in which the polymer is replaced with xanthan gum, the gel crossover point is greatly increased so as to preclude passage through a porous applicator. The presence of powders that had passed through a porous applicator were detected using plane- and cross-polarized microscopy, as further described in Example 6 herein below.

### Example 6: Evaluation of Powder Penetration Through Porous Applicator

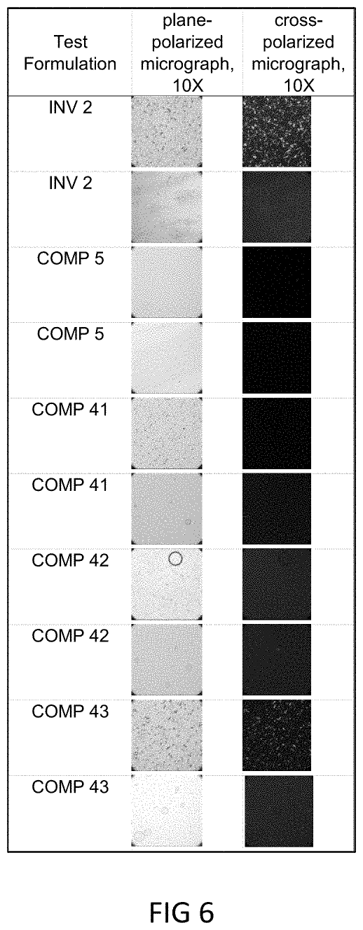

The presence of powders that had passed through the porous applicator were detected using plane- and cross-polarized microscopy. Table 5 lists the tested compositions and FIG. 6 shows cross- and plane-polarized micrographs showing bulk powder and porous applicator penetration results. All tested compositions include beta hydroxy acid (salicylic acid present at 0.5% in addition to the listed ingredients in a water and alcohol solvent (20% alcohol). In the experiment, an applicator as described herein was assembled and the retainer filled with the tested composition, followed by application via contacting the porous applicator to a surface and subsequent detection of any dispensed composition onto the surface. Each sample was evaluated twice, one sample being viewed as a bulk sample, and one sample being scanned after passage through a porous applicator as described herein. Experiment comparatives are based on the tested inventive composition with variation only in the powder content.

TABLE 5
Powder penetration through porous applicator

| | % powder in formula | | | |
|---|---|---|---|---|
| | | Micro- | | |
| FOR- | Per- | crystalline | Silica | |
| MULA # | lite | cellulose | silylate | Condition of test |
| INV 2 | 0.8 | 1.6 | 0.25 | bulk formula |
| INV 2 | 0.8 | 1.6 | 0.25 | formula that has penetrated |
| | | | | through porous applicator |
| COMP 5 | 0 | 0 | 0 | bulk formula |
| COMP 5 | 0 | 0 | 0 | formula that has penetrated |
| | | | | through porous applicator |
| EXP 41 | 0 | 0 | 0.25 | bulk formula |
| EXP 41 | 0 | 0 | 0.25 | formula that has penetrated |
| | | | | through porous applicator |
| EXP 42 | 0.8 | 0 | 0 | bulk formula |
| EXP 42 | 0.8 | 0 | 0 | formula that has penetrated |
| | | | | through porous applicator |
| EXP 43 | 0 | 1.6 | 0 | bulk formula |
| EXP 43 | 0 | 1.6 | 0 | formula that has penetrated |
| | | | | through porous applicator |

Referring now to FIG. 6, as shown in the micrographs under test conditions listed in Table 5, the inventive composition INV 2 was shown to pass through the porous applicator. COMP 5, which lacks any powder, serves as a negative control. Comparative compositions EXP 41, EXP 42 and EXP 43 are each based on INV 2 and each contains only one of the powders from the INV 2, and as shown in the micrographs in FIG. 6, the powders were able to pass through the pores. These results demonstrate that the inventive compositions herein having the desirable gel properties are suited for passage through the porous applicator, whereas results reported herein for certain of the comparative compositions demonstrated a lack of the gel crossover properties needed to transition through the applicator as a stable gel dispersion of powder.

### Example 7. Evaluation of Sweat Resistance of Inventive and Comparative Compositions

TABLE 6
Sweat resistance

| | Sweat resistance - performance index compared | | | | | |
|---|---|---|---|---|---|---|
| | to positive and negative references | | | | | |
| | | | 6 min | | 12 min | |
| FORMULA | pre-spray | | post-spray | | post-spray | |
| # | average | error | average | error | average | error |
| INV 2 | 47.56 | 3.76 | 41.49 | 1.56 | 32.90 | 3.94 |
| COMP 5 | 16.72 | 6.41 | 5.84 | 5.75 | −12.97 | 11.87 |
| NEG CTRL | 0 | 0 | 0 | 0 | 0 | 0 |
| POS CTRL | 100 | 0 | 100 | 0 | 100 | 0 |

| performance index | indication |
|---|---|
| >=100 | better than positive reference |
| 0-100 | better than negative reference and worse than |
| | positive reference |
| <=0 | worse than negative reference |

Negative Control Test Composition for Sweat Study

COMPOSITION FORMULA NEGATIVE CONTROL (NO/LOW
SWEAT RESISTANCE: Normaderm Placebo)

| | W/W % |
|---|---|
| RM INCI | Formula |
| WATER | 67.85 |
| GLYCERIN | 7 |
| PROPYLENE GLYCOL | 2 |
| AMMONIUM POLYACRYLOYLDIMETHYLTAURATE | 1 |
| DIMETHICONE | 7 |
| PEG-12 DIMETHICONE | 0.7 |
| DIMETHICONE | 4 |
| BIOSACCHARIDE GUM-1 | 2 |
| CAPRYLYL GLYCOL | 0.4 |
| MENTHOL | 0.05 |
| ALCOHOL DENAT. | 8 |

Positive Control Test Composition for Sweat Study

COMPOSITION FORMULA POSITIVE CONTROL (COMPLETE
SWEAT RESISTANCE; Normaderm Placebo)

| | |
|---|---|
| WATER | 65.85 |
| GLYCERIN | 7 |
| PROPYLENE GLYCOL | 2 |
| AMMONIUM POLYACRYLOYLDIMETHYLTAURATE | 1 |
| DIMETHICONE | 7 |
| PEG-12 DIMETHICONE | 0.7 |
| DIMETHICONE | 4 |
| BIOSACCHARIDE GUM-1 | 2 |
| SILICA SILYLATE | 2 |
| CAPRYLYL GLYCOL | 0.4 |
| MENTHOL | 0.05 |
| ALCOHOL DENAT. | 8 |

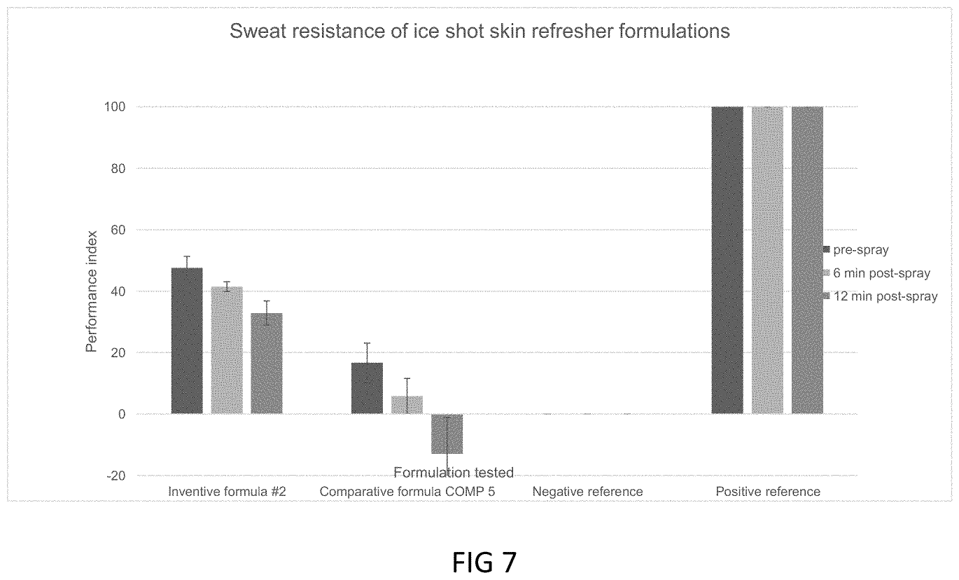

Sweat resistance was assessed in vitro using a Normaderm placebo with no powders as the negative reference and a Normaderm formula with 2% silica silylate as a positive reference, having the composition formulae as listed above. Three replicates were tested for each formulation, and the data shown sets forth the averages and errors obtained. The sweat resistance data are shown above in Table 6 and in FIG. 7. Drawdowns of various prototypes and the references were sprayed with artificial sweat (Oleic acid, water, oleth-10) and measured with a glossmeter. This invention (e.g., INV 2) was found to resist sweat about 50% as well as the POS CTRL, while the same formula sans powders (COMP 5) only resisted about 8% as well as the POS CTRL.

### Example 7: Qualitative Study of Inventive Composition by Consumers

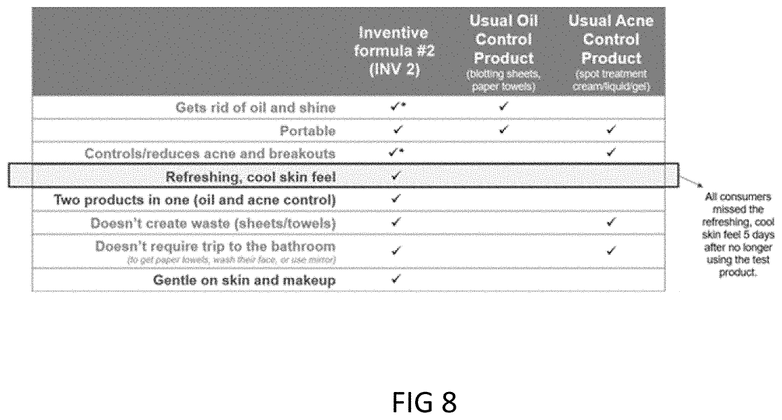

A qualitative study of 20 US consumers, 50% men and 50% women, clearly showed this invention (e.g., INV 2) eliminates oil and shine, controls/reduces acne and breakouts, has a refreshing, cool skin feel, and is gentle on skin and makeup. A summary of consumer comments from the study are shown in FIG. 8, which addresses study including an inventive composition and commercial oil control and acne control products.

A qualitative study of US consumers showed that, compared to the consumers' usual oil control products, such as blotting sheets and paper towels, the cosmetic composition according to this invention additionally controls/reduces acne and breakouts, provides a refreshing, cool skin feel, acts as two products in one (oil and acne control), doesn't create waste, doesn't require a trip to the washroom, and is gentle on skin and makeup. The study also showed that, compared to the consumers' usual acne control products which include spot treatment creams/liquids/gels, consumers found that the cosmetic composition according to this invention eliminates oil and shine, provides a refreshing, cool skin feel, acts as two products in one (oil and acne control), and is gentle on skin and make up. With a similar concentration of oil absorbing powders and ethanol, this inventive cosmetic composition is capable of both controlling shine and providing a refreshing, cool skin feel without disturbing the user's make up with the key benefit of treating acne and other skin blemishes through the delivery of salicylic acid.

While the disclosure has been described with reference to described embodiments, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one” or “one or more” as used herein, means that there may be one, two, three or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

The terms “free” and “devoid” indicates that no reliably measurable excluded material is present in the cosmetic composition, typically 0% by weight, based on the total weight of the cosmetic composition. The term “essentially free” means that, while it prefers that no excluded material is present in the cosmetic composition, it is possible to have very small amounts of the excluded material in the cosmetic composition of the invention, provided that these amounts do not materially affect the advantageous properties of the cosmetic composition. In particular, “essentially free” means that excluded material can be present in the cosmetic composition at an amount of less than about 0.1% by weight, based on the total weight of the cosmetic composition.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated. As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc. As used herein a range of ratios is meant to include every specific ratio within, and combination of subranges between the given ranges.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1. A cosmetic composition, comprising:
a hydroalcoholic gel dispersion, comprising
(a) at least one beta hydroxy acid selected from -n-octanoylsalicylic acid, salicylate, sodium salicylate, willow extract, beta hydroxybutanoic acid, tropic acid, and trethocanic acid, the beta hydroxy acid present in the cosmetic composition in an amount that is up to about 2.0%;
(b) a hydroalcoholic gel, comprising:
(i) at least one C2-C5 alcohol, and
(ii) at least one polymer that includes acryloyldimethyltaurate/steareth-25 methacrylate crosspolymer; and
(c) at least one powder that includes silica silylate, and optionally one or more additional powders selected from inorganic powders, organic powders, and silica;
wherein the at least one beta hydroxy acid is dissolved in the hydroalcoholic gel and the at least one powder is dispersed in the hydroalcoholic gel.

2. The cosmetic composition according to claim 1, wherein the at least one beta hydroxy acid is salicylic acid.

3. The cosmetic composition according to claim 1, wherein at least one beta hydroxy acid comprises salicylic acid present in the composition in the range from about 0.2% to about 2%, by weight, based on the total weight of the composition.

4. The cosmetic composition according to claim 1, wherein the at least one C2-C5 alcohol is selected from ethanol, propanol, butanol, pentanol, isopropanol, isobutanol and isopentanol.

5. The cosmetic composition according to claim 1, wherein the at least one C2-C5 alcohol comprises denatured alcohol present in the composition in the range from about 5% to about 20%, by weight, based on the total weight of the composition.

6. The cosmetic composition according to claim 1, wherein the pH of the cosmetic composition is in the range from about 3.9 to about 4.5.

7. The cosmetic composition according to claim 1, comprising at least one additional powder selected from perlite and microcrystalline cellulose.

8. The cosmetic composition according to claim 1, comprising additional powders comprising from perlite and microcrystalline cellulose.

9. The cosmetic composition according to claim 1, wherein the cosmetic composition excludes one or more powders comprising Nylon-12, polymethyl methacrylate, polyethylene beads, and powders which are considered microplastics.

10. The cosmetic composition according to claim 1, wherein each of the silica silylate and any one or more additional powders is present in the cosmetic composition in an amounts ranging from about 0.005% to about 10%, by weight based on the weight of the composition.

11. The cosmetic composition according to claim 1, wherein the cosmetic composition excludes at least one or all of polyacrylate crosspolymer 6, and an acrylates/C10-30 Alkyl acrylate crosspolymer.

12. The cosmetic composition according to claim 1, wherein the cosmetic composition has a gel crossover point ranging from about 35% to about 85% strain.

13. The cosmetic composition according to claim 1, wherein the cosmetic composition has a gel crossover point which allows it to suspend the at least one powder and pass through a porous applicator with shear.

14. The cosmetic composition according to claim 15, wherein the cosmetic composition has a gel crossover point ranging from about 35% to about 85% strain.

15. A cosmetic composition, comprising: (a) salicylic acid present in an amount in the range from about 0.2% to about 2%, and an (b) aqueous hydroalcoholic gel dispersion, comprising: (i) denatured alcohol in an amount that is about 20% by weight, (ii) perlite present in the cosmetic composition in an amount that ranges from about 0.5% to about 1% by weight, microcrystalline cellulose present in the cosmetic composition in an amount that ranges from about 1.4% to about 2% by weight, and silica silylate present in the cosmetic composition in an amount that ranges from about 0.2% to about 0.3% by weight, and (iii) acryloyldimethyltaurate/steareth-25 methacrylate crosspolymer present in the cosmetic composition in an amount that ranges from about 0.9% to about 2.4% by weight.

16. A cosmetic dispensing system, comprising:
a cosmetic composition that has a gel crossover point suitable for dispensing through a porous applicator, the cosmetic composition comprising:
a hydroalcoholic gel dispersion, comprising
(a) at least one beta hydroxy acid selected from -n-octanoylsalicylic acid, salicylate, sodium salicylate, willow extract, beta hydroxybutanoic acid, tropic acid, and trethocanic acid, the beta hydroxy acid present in the cosmetic composition in an amount that is up to about 2.0%;
(b) a hydroalcoholic gel, comprising:
(i) at least one C2-C5 alcohol, and
(ii) at least one polymer that includes acryloyldimethyltaurate/steareth-25 methacrylate crosspolymer; and
(c) at least one powder that includes silica silylate, and optionally one or more additional powders selected from inorganic powders, organic powders, and silica;
wherein the at least one beta hydroxy acid is dissolved in the hydroalcoholic gel and the at least one powder is dispersed in the hydroalcoholic gel; and
a dispenser that includes a container that includes a retainer and an absorbent component disposed within the retainer, and a porous applicator that is in contact on a first side with the absorbent component in the retainer, and is exposed on a second side for contacting keratinous tissue to be treated,
wherein the cosmetic composition is absorbed within the absorbent component and retained in the retainer, which retains the cosmetic composition in a dispersed gel state until dispensed; and
wherein the dispenser optionally comprises a cap for covering the porous applicator when the dispensing system is not in use.

17. The cosmetic dispensing system according to claim 16, wherein the porous applicator of the cosmetic dispensing system includes one or more of:
a. on at least the exposed surface intended for contacting skin, a plurality of pores having an average size in a range from about 30 to about 200 microns;
b. on at least the first surface intended for contacting the sponge containing the cosmetic composition in the retainer, a plurality of pores having an average size in a range from about 90 to 200 microns;
c. from the first surface to the second surface throughout the porous applicator, a plurality of pores having an average size in a range from 1 to about 1000 microns;
d. the porosity through the porous material from the first surface to the second surface is uniform and continuous;
e. the porosity through the porous material from the first surface to the second surface is variable from larger pores at the first surface to smaller pores at the second surface; and
f. the porosity through the porous material from the first surface to the second surface is variable from smaller pores at the first surface to larger pores at the second surface.

18. A method for enhancing the appearance of acne prone skin, comprising: providing a dispensing system comprising: a cosmetic composition that has a gel crossover point suitable for dispensing through a porous applicator, and a dispenser that includes a container that includes a retainer and an absorbent component disposed within the retainer, and a porous applicator that is in contact on a first side with the absorbent component in the retainer, and is exposed on a second side for contacting keratinous tissue to be treated,
the cosmetic comprising:
a hydroalcoholic gel dispersion, comprising
(a) at least one beta hydroxy acid selected from -n-octanoylsalicylic acid, salicylate, sodium salicylate, willow extract, beta hydroxybutanoic acid, tropic acid, and trethocanic acid, the beta hydroxy acid present in the cosmetic composition in an amount that is up to about 2.0%;
(b) a hydroalcoholic gel, comprising:
(i) at least one C2-C5 alcohol, and
(ii) at least one polymer that includes acryloyldimethyltaurate/steareth-25 methacrylate crosspolymer; and
(c) at least one powder that includes silica silylate, and optionally one or more additional powders selected from inorganic powders, organic powders, and silica;
wherein the at least one beta hydroxy acid is dissolved in the hydroalcoholic gel and the at least one powder is dispersed in the hydroalcoholic gel; and
wherein the dispenser containing the cosmetic composition is used to apply the composition to skin that is prone to acne by utilizing the dispenser to apply the cosmetic composition by contacting the porous applicator to skin using a dabbing motion to dispense a light coating of the cosmetic composition onto select areas of the skin in dispensed doses that are in an amount that is from about 0.01 mg to about 100 mg per application stroke.
